(19) United States
(12) Patent Application Publication
(10) Pub. No.: US 2007/0265211 A1
(43) Pub. Date: Nov. 15, 2007

(54) Novel composition and method effective in inhibiting the atherogenic process

(76) Inventors: Matthias Rath, Bocholtz (NL); Aleksandra Niedzwiecki, San Jose, CA (US); Vadim Ivanov, Castro Valley, CA (US); Waheed Roomi, Sunnyvale, CA (US); John Cha, San Mateo, CA (US)

Correspondence Address: INHOUSE IP, 280 COLORADO AVE, PALO ALTO, CA, 94301, US

(21) Appl. No.: 11/432,830

(22) Filed: May 12, 2006

(51) Int. Cl. A61K 31/714; A61K 31/7076; A61K 31/7048; A61K 31/525; A61K 31/205

(52) U.S. Cl. 514/27; 514/47; 514/52; 514/250; 514/251; 514/474; 514/350; 514/562; 514/500; 514/554; 514/559; 514/564

(57) ABSTRACT

The present invention provides biochemical compositions effective in inhibiting an atherogenic process, comprising ascorbic acid, lysine, magnesium, cysteine, pyridoxine HCL, riboflavin, folic acid, cyanocobalamin vitamin B12, S-Adenosyl-L-Methionine (SAMe), choline bitartrate, copper glycinate, epigallocatechin gallate, quercetin, asiatic acid, and pycnogenol.

### FIELD OF THE INVENTION

This invention relates to compositions effective in inhibiting an atherogenic process or atherosclerosis, which is conceptually defined as the result of a multitude of interactive cascade of injurious stimuli and the healing responses of the arterial wall, generally in the presence of a hyperlipidemic environment and more specifically in the presence of a low density lipoproteins (LDLs). More particularly, this invention relates to a composition effective in inhibiting growth of smooth muscle cell, invasion of extracellular matrix by smooth muscle cell and blood monocytes, and deposition of extracellular matrix by smooth muscle cells.

### BACKGROUND OF THE INVENTION

Atherosclerosis and its associated vascular complications are the principal causes of cardiovascular and cerebrovascular diseases leading to myocardial infarction and stroke, respectively. Every year over 12 million people worldwide die of the results of atherosclerosis, heart infarctions, and strokes. According to the American Heart Association's 2004 Heart and Stroke Statistical Update, over 64 million people worldwide suffer from cardiovascular disease, which has been the leading cause of death in the US for decades.

The formation of an atherosclerotic lesion as a result of the atherogenic process is associated with drastic behavioral modifications by arterial wall smooth muscle cells (SMC), including: massive migration of SMC from the vascular medial to the intima layer and dedifferentiation of SMC to proliferating phenotype. These events facilitate vascular wall thickening and monocyte recruitment from blood, and lead to progression of the atherogenic cascade. In addition, vascular changes in atherosclerosis involve neointimal thickening resulting from the increased deposition of extracellular matrix proteins by smooth muscle cells that migrate and proliferate in the affected blood vessel areas. Various patho-physiologic events can aggravate this process, such as inflammation, oxidative processes accompanying low-density lipoprotein and lipoprotein(a) deposition, and intracellular membrane mediated events, such as changes in protein kinase C activity. Various matrix components also affect cellular proliferation, differentiation and expression of specific genes.

Taking into account that natural occurrence of atherosclerosis is limited to humans, primates and guinea pigs (species not producing vitamin C) and it is most frequently manifested in specific mechanistically stressed areas of the coronary arteries we have been focusing on vascular stability as a critical factor in atherosclerosis. Rath and Pauling proposed that chronic sub clinical vitamin C deficiency has destabilizing effect on vascular wall structure and function leading to deposition of lipoprotein(a) and fibrinogen/fibrin in the vascular wall and triggering other physiological changes characteristic of atherosclerosis. The critical role of ascorbic acid in the stability of vascular wall stems from the fact that this compound is necessary for the synthesis and enzymatic hydroxylation of proline and lysine residues in collagen molecules. In this context Nakata and Maeda (Circulation 2002; 105:1485-1490) has shown that a loss of vitamin C production in mice, a species which normally synthesizes vitamin C, resulted in structural changes in the coronary arteries resembling early atherosclerosis. A dose-dependent decreased proliferation of the vascular smooth muscle cells (VSMC) from guinea-pig aorta in the presence of 0.5-2.0 mM ascorbate through direct and matrix-mediated effects was observed. In addition, ascorbate has been shown to induce SMC differentiation, which results in a reduction in cell growth important in curbing atherosclerotic plaque development. In addition to ascorbate, several other nutrients are essential in optimizing vascular connective tissue structure and function, such as lysine, proline, copper, manganese and others. Additionally, a number of studies have shown cardio-protective effects of green tea consumption.

Naturally occurring compounds demonstrate a wider spectrum of biological activity and fewer side effects than synthetic drugs and a mixture of natural compounds often produces synergistically enhanced therapeutic effects. This reasoning prompted us to investigate whether a mixture of nutrients, including ascorbic acid, lysine, cysteine and plant-derived polyphenolics: epigallocatechin gallate from green tea extract, quercetin, rutinoside (rutin) and asiatic acid from Gotu Kola extract, would demonstrate anti-atherogenic effects using the model of cultured vascular smooth muscle cell, vascular endothelial cells and monocytes.

There is a long felt need to provide a safe and effective nutrient pharmaceutical composition and method for the treatment of atherosclerosis that do not have side effects.

There is yet another need for compounds and substances in the retardation of development of atherosclerosis, inhibition of growth of smooth muscle cell, inhibition of invasion of extracellular matrix by smooth muscle cell using low cost non-drug substances and compounds instead of expensive drugs.

### SUMMARY OF THE INVENTION

It is an object of the present invention to provide biochemical compositions effective in inhibiting an atherogenic process.

The atherogenic process include of the growth of smooth muscle cell and the invasion of extracellular matrix by smooth muscle cell.

Accordingly, the present invention provides biochemical compositions effective in prevention and treatment resulting in inhibiting an atherogenic process, comprising ascorbic acid, lysine, magnesium, cysteine, pyridoxine HCL, riboflavin, folic acid, cyanocobalamin vitamin B12, S-Adenosyl-L-Methionine (SAMe), choline bitartrate, copper glycinate, epigallocatechin gallate, quercetin, asiatic acid, and pycnogenol.

Alternatively, the present invention provides biochemical compositions effective in prevention and treatment resulting in inhibiting an atherogenic process, comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

Preferably, the present invention provides biochemical compositions effective in prevention and treatment resulting in inhibiting an atherogenic process comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

Additionally, the present invention provides a method in prevention and treatment resulting for retarding the progression of atherosclerosis in a mammal comprising the step of administering to the mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol. Preferably, the composition comprises 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

Alternatively, the present invention provides a method for prevention and treatment resulting inhibiting the invasion of extracellular matrix by smooth muscle cell in a mammal comprising the step of administering to the mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol. Preferably, the composition comprises 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

Optionally, the present invention provides a method for in prevention and treatment resulting inhibiting the growth of smooth muscle cell in a mammal comprising the step of administering to the mammal an effective amount of the composition comprising ascorbic acid, lysine, magnesium, cysteirie, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol. Preferably, the composition comprises 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

Alternatively, the present invention provides a method in prevention and treatment resulting inhibiting an atherogenic process in a mammal comprising the step of administering to the mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol. Preferably, the composition comprises 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

Alternatively, the present invention provides a method of prevention and treatment resulting in optimization of the composition of connective tissue in mammals.

More preferably, the compositions may be administered orally, intravenously, or parenterally.

### BRIEF DESCRIPTION OF THE DRAWING

FIG. 1 shows effects of various concentrations of the mixture of the composition of the invention on growth of human aortic smooth muscle cell. Cell growth rate was evaluated by incorporation of [3H]-thymidine into cellular DNA during last 24 hours of the experiment.

FIG. 2a shows effects of various concentrations of the mixture of the composition of the invention on secondary smooth muscle cell growth (3H-thymidine incorporation) on smooth muscle cell -deposited extra cellular matrix.

FIG. 2b shows aortic smooth muscle cell attachment to extra cellular matrix deposited by smooth muscle cell under supplementation with mixture of the composition of the invention.

FIG. 3 shows U937 and aortic smooth muscle cell invasion through smooth muscle cell—extra cellular matrix layer. Effects of smooth muscle cell—extra cellular matrix formation under treatment with 50 mcg/ml mixture of the composition of the invention. FIG. 4a shows Collagen types I and IV deposition by aortic smooth muscle cell under treatment with mixture of the composition of the invention (100 mcg/ml) or ascorbate (100 mcM) for 3 days.

FIG. 4b shows Collagen types I and IV deposition by aortic endothelium cells under treatment with mixture of the composition of the invention (100 mcg/ml) or ascorbate (100 mcM) for 3 days.

FIG. 5a shows effects of mixture of the composition of the invention or Ascorbate supplementation for 3 days on Collagen types IV:I ratio in extra cellular matrix deposited by cultured human aortic smooth muscle cells.

FIG. 5b shows effects of mixture of the composition of the invention or Ascorbate supplementation for 3 days on Collagen types IV:l ratio in extra cellular matrix deposited by human aortic endothelial cells.

FIG. 6a shows Chondroitin Sulfate and Heparan Sulfate deposition by aortic smooth muscle cell under treatment of the mixture of the composition of the invention (100 mcg/ml) or ascorbic acid (100 nM.)

FIG. 6b shows Chondroitin Sulfate and Heparan Sulfate deposition by aortic Endothelial cells under treatment with mixture of the composition of the invention (100 mcg/ml) or ascorbic acid (100 nM.)

FIG. 7a shows effects of mixture of the composition of the invention or Ascorbate supplementation for 3 days on Glycosaminoglycans Ratio in extra cellular matrix deposited by aortic smooth muscle cell.

FIG. 7b shows effects of mixture of the composition of the invention or Ascorbate supplementation for 3 days on Glycosaminoglycan Ratio in extra cellular matrix deposited by aortic endothelial cells.

FIG. 8 shows effects of Bioflavonoids and ascorbic acid on Heparan Sulfate content in extra cellular matrix deposited by human aortic smooth muscle cell.

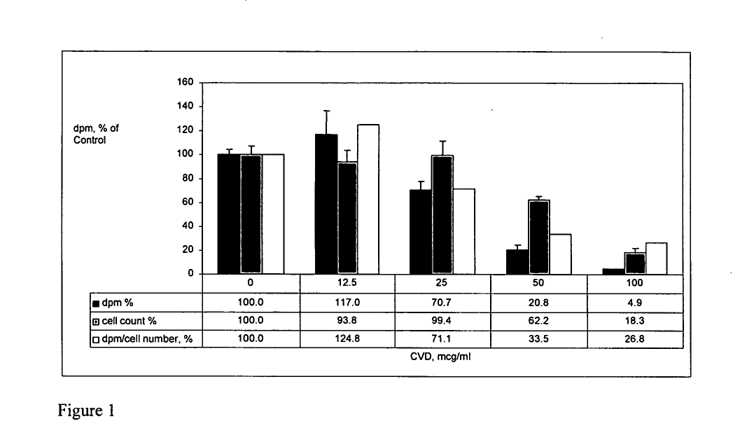

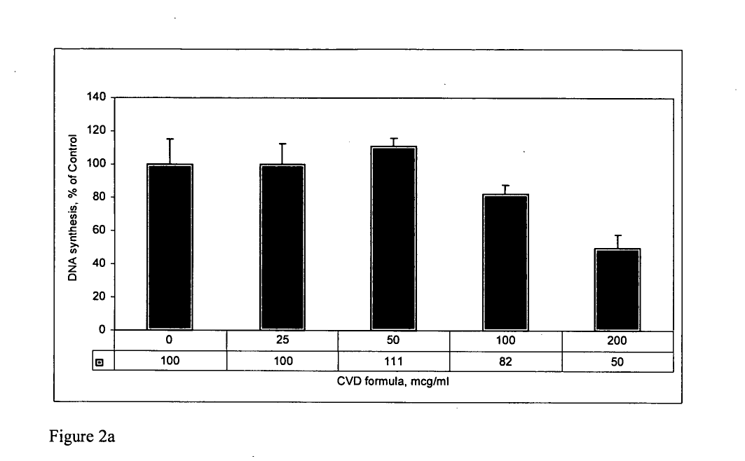

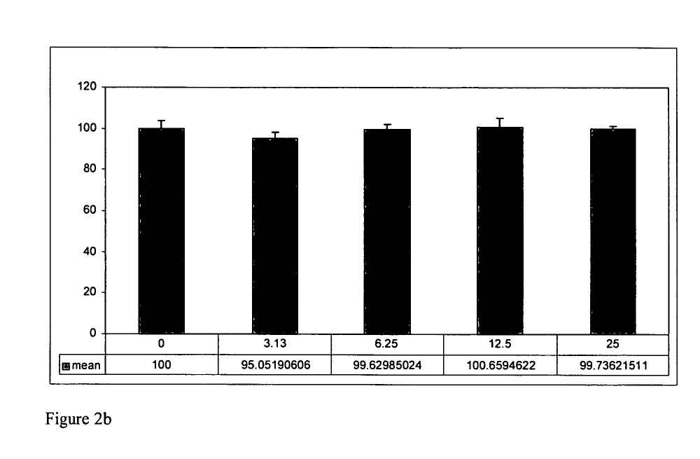

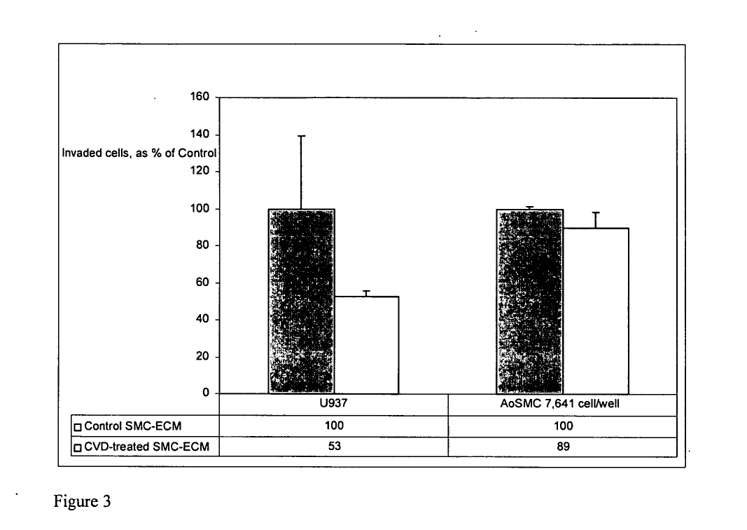

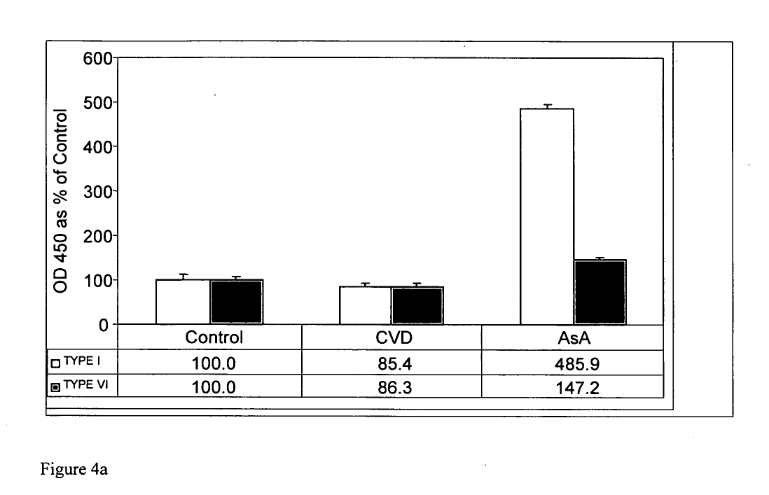

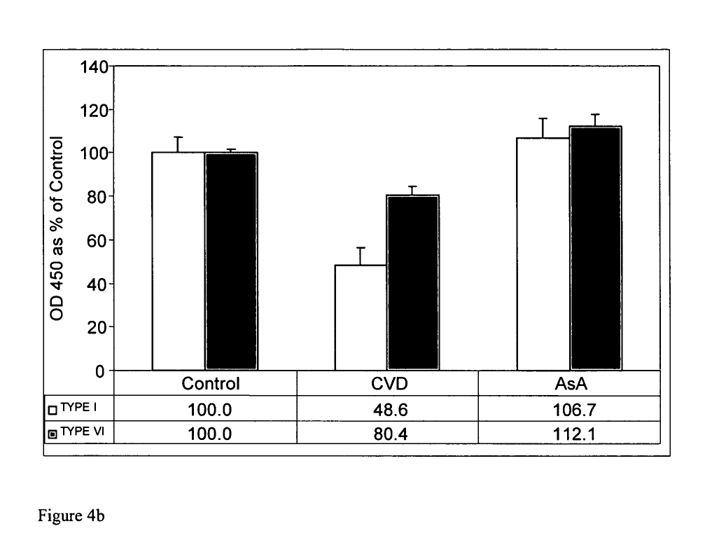

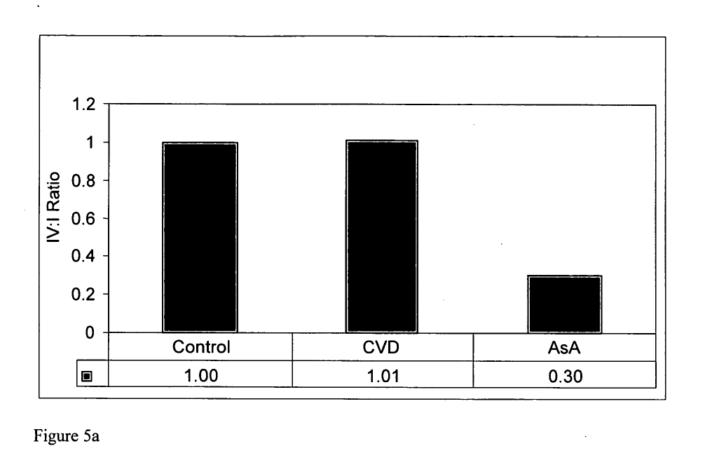

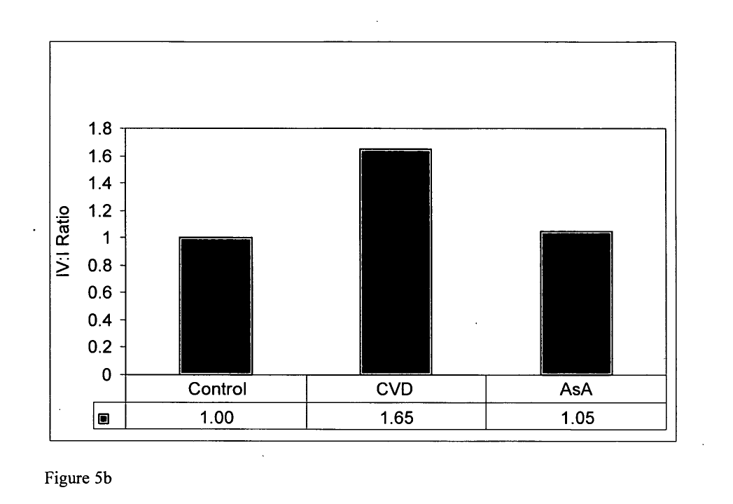

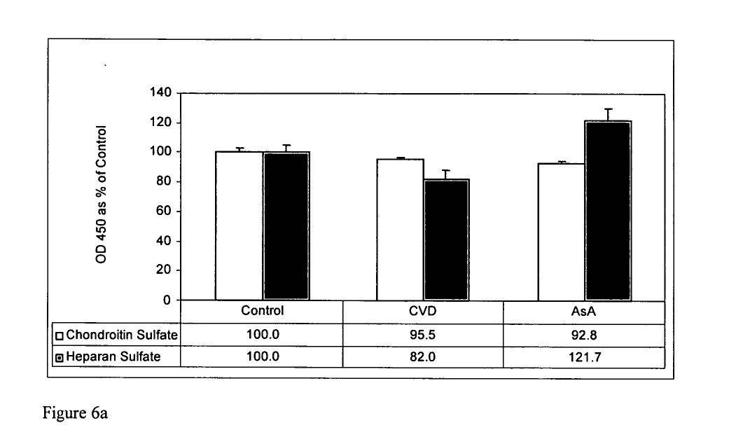

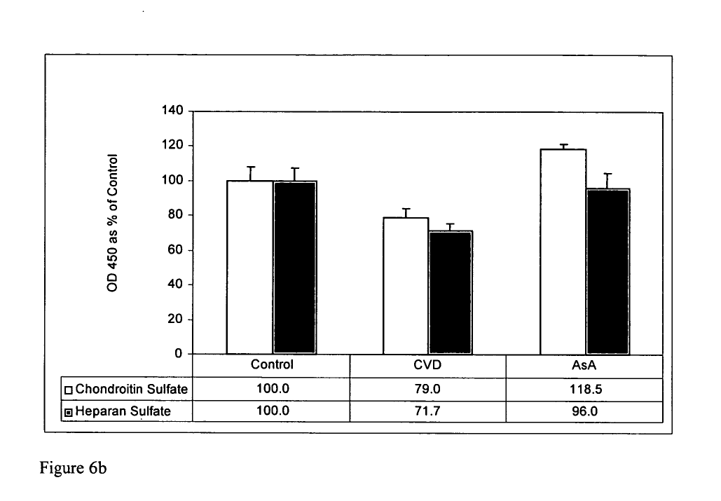

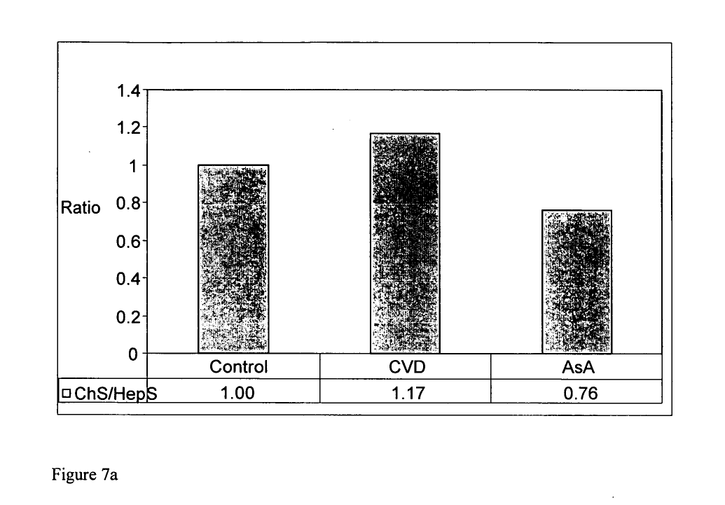

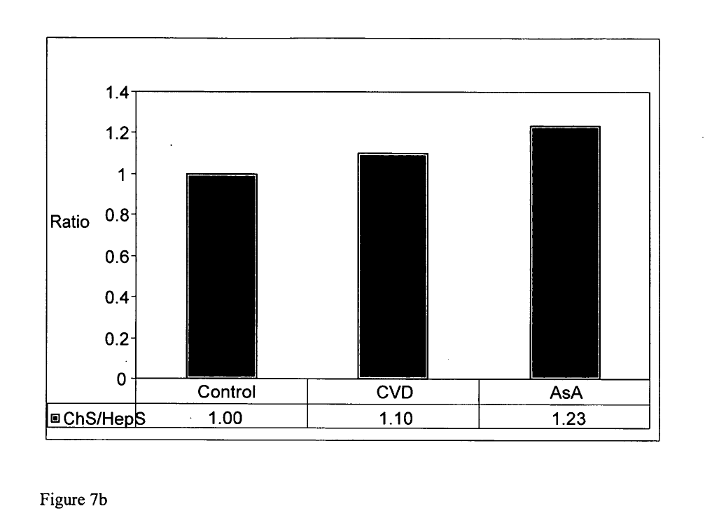

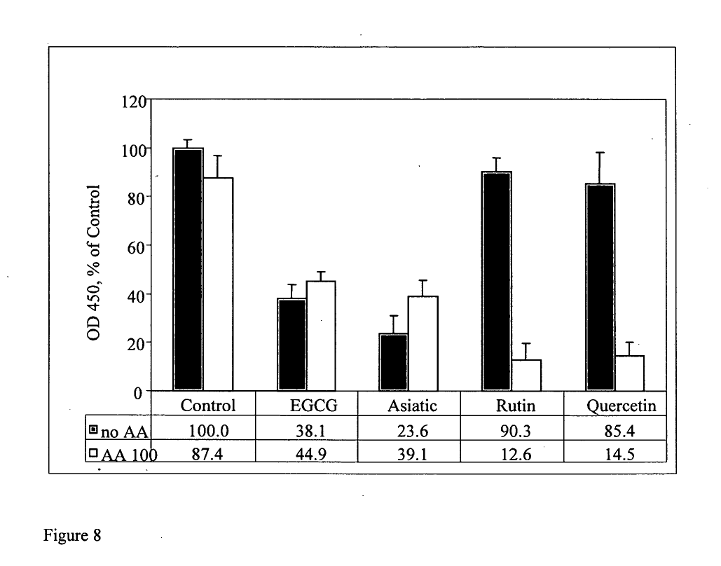

### DETAILED DESCRIPTION OF THE INVENTION

Tissue culture plastics were obtained from Becton Dickinson, USA. Tissue culture supplies (growth media, antibiotics, and trypsin-EDTA) were obtained from Life Technologies, USA. Fetal bovine serum (FBS) was from BioWhittaker (Walkersville, Md., USA). Scintillation fluid BetaBlend and [methyl-3H] Thymidine (25 Ci/mole) were from ICN Biomedicals (Costa Mesa, Calif., USA). L-ascorbic acid, bovine serum albumin (fraction V) (BSA), and other chemicals were from Sigma-Aldrich, USA.

Human aortic smooth muscle cells (SMC, obtained from Clonetics) were cultured in DMEM (Dulbecco's modified Eagle's medium), supplemented with 10% fetal bovine serum, penicillin (100 μg/ml) and streptomycin (100 μg/ml) at 37° C. in a humidified atmosphere containing 5% CO₂, and were split 1:3 to 1:5 upon reaching the confluence. SMC at passages 5-8 were used in experiments. Human aortic endothelial cells (EC, obtained from Clonetics) were cultured in Clonetics-specified Endothelial Cell Medium, supplemented with 5% fetal bovine serum, penicillin (100 mg/ml) and streptomycin (100 mg/ml) at 37° C. in a humidified atmosphere containing 5% CO2, and were split 1:3 to 1:5 upon reaching the confluence. EC at passages 5-8 were used in experiments.

SMC proliferation was assayed by [3H]-thymidine incorporation into cellular genetic material. Cells were plated in 24-well plates at a density of 10,000 cells per cm² in 0.5 ml of DMEM supplemented with 2% FBS. The attached cells were supplied every 24 hours with fresh growth medium plus additions, as specified in the protocols. Test agents included the nutrient mixture and individual components. A stock solution of the nutrient mixture was prepared daily immediately before addition to cell cultures by solving in DMEM to a concentration of 10 mg/ml, vigorously vortexing for 1 minute under high speed, and filtering through a 0.2 μm sterile filter. Cell proliferation was measured 3 days later by the addition of 1 pCi/ml [3H]-thymidine to the cell culture for the last 4 hours of the experiment. Cells were washed three times with cold phosphate-buffered saline, pH 7.2, incubated with 10% trichloroacetic acid for 15 minutes at 4° C., washed with cold ethanol, air-dried, soluabilized in 0.5 N sodium hydroxide, and then neutralized with hydrochloric acid. Samples were mixed with scintillation fluid and counted using a liquid scintillation counter (model 6500 LS, Beckman Instruments, USA). Cellular DNA-incorporated radioactivity was expressed as d/min per well.

In some wells cells were stained with Hematoxylin/Eosin and cell nucleus were counted under microscope in standard way chosen views covering total 65% of the well area. Cell counting data expressed as cell number per well.

Cell Invasion Through SMC-ECM Layer

SMC were seeded on top of cell culture well inserts with porous plastic membrane covered with Collagen type I (pores 3 micro m in diameter) and grown in 5% FBS/DMEM until reaching confluence. Cells were supplemented with tested combination at 50 meg/ml final concentration or control medium for 7 days. Before invasion study SMC-ECM layers were washed three times with PBS.

Separate stock of proliferating SMC was metabolically labeled with 3H-thymidine (0.5 mcCi/ml) for 24 h at 37° C. in 75 sq. cm flask. Cells were washed three times with PBS, suspended by Trypsin/EDTA treatment, and resuspended in serum-free DMEM without any supplementation. Cells were diluted to concentration 100,000 cells per ml and added to upper portion of the inserts. Lower chambers were supplemented with 10 ng/ml fibroblasts growth factor in serum-free DMEM to initiate the invasion process. After incubation for 24 h at 37° C. inserts were removed from the wells, washed three times with PBS, top side of the insert membrane was wiped clean from cells with cotton swipes, number of cells invaded to the lower side of membrane was counted according to radioactive count in scintillation counter.

Human monocytic cells (line U937) grown in suspension 5% FBS/RPMI-1640 were used for invasion studies similarly to SMC with the following exceptions: washing of U937 cell suspension was done by sedimentation at centrifugation, final cell concentration for invasion study was 500,000 cell/ml and monocyte chemoattracting protein 1 was used as chemoattractant.

SMC Growth On Pre-Deposited Extracellular Matrix (ECM)

SMC were grown in 24-well plates in 5% FBS/DMEM until reaching confluence. Cells were supplemented with tested combination at 50 mcg/ml final concentration or control medium for 7 days. To remove cells and expose ECM cell were washed three times with PBS and incubated consecutevely with 0.5% Triton X100/PBS and 0.1M NH40H/PBS for 3 min each at RT to remove cells and expose underlying ECM.

Fresh proliferating SMC culture was seeded on top of exposed ECM in 5%FBS/DMEM. After cell attachment for 3-4 hours, medium was changed for a new one and cells were incubated for 72 h at 37° C. Cell proliferation was assayed by addition of 0.5 mcCi 3H-thymidine for the last 4 h of incubation and cellular DNA synthesis was assayed as described above.

In some wells cells were assayed for attachment efficiency by incubating cells for 4 hours in serum-free medium containing 0.5 mg/ml MTT. At the end of incubation cell media was replaced with DMSO, and extracted formazan salt were measured by optical density at 550 nm. There was no difference between different ECM in SMC attachment efficiency.

ECM Components Assay

SMC or EC were grown in 96-well plates in 5% FBS/DMEM or 5%FBS/ECM, respectively, until reaching confluence. Supplementations of tested compounds were made over three or five days, after that ECM was prepared as described above. Measurements of ECM components were done in ELISA-like assay. Wells with exposed ECM were incubated with appropriate dilution of primary specific antibody in 1% BSA/PBS for 2 h at RT, washed three times with 0.1% BSA/PBS, followed by 1.5 h incubation at RT with appropriate dilution of secondary antibody conjugated with horse raddish peroxidase. TMB substrate was developed for 20 min at RT in the wells after repeated washing cycle and amounts of ECM component of interest was found to be proportional to otrical density at 450 nm.

Extracellular matrix plays a significant role in arterial wall tissue integrity and behavior of tissue resident cells. Development of atherosclerotic lesion in arterial wall is believed to be associated with significant changes in structure and properties of ECM: increase in overall volume, increased total collagen content with specific replacement of Collagen type IV by Collagen type I. There is a significant increase in total content of sulfated glycosaminoglycans with specific depletion of chondroitin sulfate and increased accumulation of heparan sulfate. These changes lead to developing a weak amourphous extracellular matrix causing a formation of weak porous spots in arterial walls. This in turn significantly contributes to initiation or aggravatation of such atherosclerotic processes as recruiting and retention cells from blood lumina and surrounding tissues; retention, overproduction and autocrine effects of numerous growth factors and inflammatory cytokines, retention and subsequent oxidative modification of blood plasma low density lipoprotein and consequent intra- and extracellular lipid accumulation. Weakened ECM contributes to atherosclerotic plaque rupture triggering platelet adhesion and activation and thrombus formation. Thus overall reduction of the ECM volume produced by arterial wall resident cells: SMC and EC, accompanied by favorable switch in particular ECM component distribution pattern is one of the therapeutic targets in preventing and managing atherosclerotic process.

Lysine may include lysine salts such as hydroxylysine and hydroxylysine salts. Typically, the L-lysine is administered in a daily dose of 5 to 208 mg/kg, and preferably 11 mg/kg. L-lysine may be administered orally in a dosage form once, twice or three times a day. For an average individual weighing 72 kg, the recommended total amount of lysine per daily administration is 350 mg to 15 grams, and more preferably approximately 800 mg.

Ascorbate compounds may include ascorbic acid, ascorbate salts and its derivatives thereof. As used herein, ascorbic acid and vitamin C are used interchangeably and include calcium ascorbate, magnesium ascorbate or ascorbyl palmitate. Typically, ascorbic acid is administered in a daily dose of 7 to 139 mg/kg, and preferably 11 mg/kg. Ascorbic acid may be administered orally in a dosage form once, twice or three times a day. For an average individual weighing 72 kg, the recommended total amount of ascorbic acid per daily administration is 500 mg to 10 grams, and more preferably approximately 700 mg.

The different compounds claimed in this application can be used together in form of covalently bound compounds or as physical mixture or in any other combination.

EGCG in the form of Green tea extract may be administered in a daily dose of 5 to 208 mg/kg, and preferably approximately 7 mg/kg. EGCG may be administered orally in a dosage form once, twice or three times a day. For an average individual weighing 72 kg, the recommended total amount of EGCG per daily administration is 125 mg to 525 mg, and more preferably approximately 175 mg.

Cysteine may include cystine (dimer of cysteine) and cysteine salts thereof. Cysteine may be administered in a daily dose of 1 to 28 mg/kg, preferably, and more preferably approximately 1.5 mg/kg. Cysteine may be administered orally in a dosage form once, twice or three times a day. For an average individual weighing 72 kg, the recommended total amount of Cysteine per daily administration is 72 mg to 2 grams, and more preferably approximately 100 mg.

The present invention further provides minerals and/or trace element. Trace elements may help to catalyze the production of these macromolecules needed for connective tissues.

Magnesium may be administered in a daily dose of 0.2 to 10 mg/kg, and more preferably, approximately 0.3 mg/kg. Magnesium may be administered orally in a dosage form once, twice or three times a day in the form of magnesium ascorbate. For an average individual weighing 72 kg, the recommended total amount of magnesium per daily administration is 14 mg to 750 mg, and more preferably approximately 21 mg.

Copper may be administered a daily dose of 0.01 to 0.1 mg/kg, and preferably, approximately 0.02 mg/kg. Copper may be administered orally in a dosage form once, twice or three times a day in the form of copper glycinate. For an average individual weighing 72 kg, the recommended total amount of copper per daily administration is 0.7 mg to 7 mg, and more preferably approximately 1.5 mg.

Pyridoxine HCL may be administered a daily dose of 0.01 to 0.2 mg/kg, and more preferably, approximately 0.04 mg/kg. Pyridoxine HCL may be administered orally in a dosage form once, twice or three times a day. For an average individual weighing 72 kg, the recommended total amount of Pyridoxine HCL per daily administration is 0.7 mg to 15 mg, and more preferably approximately 3 mg.

Riboflavin may be administered a daily dose of 0.01 to 1.0 mg/kg, and preferably, approximately 0.1 mg/kg. Riboflavin may be administered orally in a dosage form once, twice or three times a day. For an average individual weighing 72 kg, the recommended total amount of Riboflavin per daily administration is 0.7 mg to 70 mg, and more preferably approximately 3 mg.

Folic Acid may be administered a daily dose of 0.001 to 0.07 mg/kg, and preferably, approximately 0.005 mg/kg. Folic Acid may be administered orally in a dosage form once, twice or three times a day. For an average individual weighing 72 kg, the recommended total amount of Folic Acid per daily administration is 0.1 mg to 5 mg, and more preferably approximately 0.4 mg.

Cyanocobalamin Vitamin B12 may be administered a daily dose of 0.05 to 2 μg/kg, and preferably, approximately 0.1 μg/kg. Cyanocobalamin Vitamin B12 may be administered orally in a dosage form once, twice or three times a day. For an average individual weighing 72 kg, the recommended total amount of Cyanocobalamin Vitamin B12 per daily administration is 3.5 μg to 150 μg, and more preferably approximately 6 μg.

SAMe may be administered a daily dose of 0.15 to 15 mg/kg, and preferably, approximately 1.5 mg/kg. SAMe may be administered orally in a dosage form once, twice or three times a day. For an average individual weighing 72 kg, the recommended total amount of SAMe per daily administration is 10 mg to 1000 mg, and more preferably approximately 100 mg.

Choline Bitartrate may be administered a daily dose of 0.25 to 25 mg/kg, and preferably, approximately 2.5mg/kg. Choline Bitartrate may be administered orally in a dosage form once, twice or three times a day. For an average individual weighing 72 kg, the recommended total amount of Choline Bitartrate per daily administration is 20 mg to 2000 mg, and more preferably approximately 180 mg.

Quercetin may be administered a daily dose of 0.1 to 15 mg/kg, and preferably, approximately 3.5 mg/kg. Quercetin may be administered orally in a dosage form once, twice or three times a day in the form of Rutin. For an average individual weighing 72 kg, the recommended total amount of Quercetin per daily administration is 10 mg to 1000 mg, and more preferably approximately 250 mg.

Asiatic Acid may be administered a daily dose of 1 to 20 mg/kg, and more preferably, approximately 5 mg/kg. Asiatic Acid may be administered orally in a dosage form once, twice or three times a day in the form of 10% Gotu Kola Extract. For an average individual weighing 72 kg, the recommended total amount of Asiatic Acid per daily administration is 70 mg to 1500 mg, and preferably approximately 350 mg.

Pycnogenol may be administered a daily dose of 0.01 to 1.0 mg/kg, and preferably, approximately 0.04 mg/kg. Quercetin may be administered orally in a dosage form once, twice or three times a day. For an average individual weighing 72 kg, the recommended total amount of Quercetin per daily administration is 0.7 mg to 70 mg, and more preferably approximately 3 mg.

Alternatively, the present invention provides biochemical compositions effective in inhibiting an atherogenic process, comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

### EXAMPLES

Formula 1

| Ingredient | Dose | |
|---|---|---|
| Lysine | 800 | mg/day |
| Ascorbic Acid | 200 | mg/day |
| Magnesium Ascorbate | 262.6 | mg/day |
| Ascorbyl Palmitate | 230 | mg/day |
| Cysteine | 100 | mg/day |
| Pyridoxine HCL | 4 | mg/day |
| Riboflavin | 3.2 | mg/day |
| Folic Acid | 2.4 | mg/day |
| Cyanocobalamin Vitamin B12 | 6 | mcg/day |
| SAMe | 100 | mg/day |
| Choline bitartrate | 180 | mg/day |
| Copper glycinate | 5.4 | mg/day |
| EGCG | 175 | mg/day |
| Quercetin | 250 | mg/day |
| Asiatic Acid | 350 | mg/day |
| Pycnogenol ® | 3 | mg/day |

The term CVD in the drawings and figures refer to the composition of the invention as formulated in Example 1 in various concentration. The results from this study demonstrated that mixture of nutrients significantly attenuated the pro-atherogenic modification of SMC physiological properties such as: increased growth rate, extracellular matrix invasiveness, and production of Extracellular matrix components.

Smooth muscle cell excessive growth in affected regions in blood vessels is believed to contribute to thickening of the arterial wall tissue and to the development of atherosclerotic plaques. Control of excessive SMC growth became one of the major strategic goals in development of anti-atherosclerotic treatment. Cultured human aortic SMC growth rate, which was estimated in this study according to the rate of cellular DNA synthesis, was significantly reduced by green tea polyphenol, epigallocatechin gallate at physiologically relevant concentration. This cell growth inhibitory effect was further enhanced when EGCG was combined with such essential nutrients, as ascorbic acid and lysine.

In general, combined effect of a combination of nutrients could be expected from multiple points of their interaction with biological system on cellular or organ and tissue levels. For instance, ascorbic acid has been demonstrated to produce cell growth inhibitory effects in different cell types, including smooth muscle cells, though effective concentrations were higher than the ones used in present study. EGCG also has been associated with cell growth inhibitory activity. It is quite possible that these two compounds can add to each other effects on cell growth when used together. In addition, ascorbic acid has been reported to be very unstable under cell culturing conditions and to degrade to dehydroascorbic acid and, further, to oxalic acid, by redox-mediated mechanisms. EGCG has been shown to produce strong antioxidant effects. It is possible that free-radical—mediated degradation of ascorbic acid can be delayed in the presence of antioxidant EGCG increasing, therefore, its effective concentration and prolongating its time of action.

Another possible point of combined biological effects of the nutrient mixture is SMC synthesis and deposition of extracellular matrix. Growth of SMC plated on pre-formed extracellular matrix or on extracellular matrix components, such as collagen type I, significantly slowed down cell growth rate. Essential amino acid L-lysine and semi-essential amino acid L-proline are key components of the collagen primary structure. Ascorbic acid is a essential cofactor for lysyl- and prolyl hydroxylases, which action supports proper folding of collagen fibrils in post-translational collagen maturation process. Ascorbic acid also has been shown to induce collagen production by cultured SMC.

Another aspect of atherogenic process, migration of arterial wall residential smooth muscle cells from vessel medium layer to intima layer, also has been addressed in this study. Thus, chemoattractant-mediated SMC migration through naturally produced extracellular matrix (Matrigel) was inhibited by the nutrient mixture in dose-dependent manner.

The critical components of this nutrient mixture include ascorbic acid and lysine, which are essential for the synthesis and optimal structure of collagen. In this aspect, ascorbic acid is a cofactor in hydroxylation of proline and lysine residues in collagen fibers important for enhanced stability and strength of the connective tissue. Lysine is the most abundant amino acid in collagen and in addition it is a natural inhibitor of plasmin induced proteolysis, which triggers MMPs activation cascade and ECM degradation process (MRATH 1992) Various studies have shown that restructuring of the vascular matrix is affected by ascorbate, pyridoxine, and L-lysine.

The results of this study suggest that tested formulation of ascorbic acid, tea phenolics, selected amino acids, Rutin, Quercetin, and Asiatic Acid is effective in retarding or slowing the development of atherosclerotic lesions by inhibiting atherogenic responses of vascular SMC to pathological stimuli. It decreased aortic SMC proliferation and their invasion through extracellular matrix.

What is claimed is:

1. A composition of biochemical substances comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol that is effective in inhibiting an atherogenic process.

2. The composition according to claim 1, wherein the ascorbic acid is selected from the group consisting of calcium ascorbate, magnesium ascorbate and ascorbyl palmitate.

3. The composition according to claim 1, wherein the folic acid is folate.

4. The composition according to claim 1, wherein the nutritional composition comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 pg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

5. The composition according to claim 1, wherein the nutritional composition comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

6. A method of prevention and treatment resulting in inhibiting an atherogenic process in mammals, comprising the step of administering to a mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol.

7. A method of prevention and treatment resulting in inhibiting growth of smooth muscle cell in mammals, comprising the step of administering to a mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol.

8. A method of prevention and treatment resulting in inhibiting invasion of extracellular matrix by smooth muscle cell in mammals, comprising the step of administering to a mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol.

9. A method of prevention and treatment resulting in retarding progression of atherosclerosis in mammals, comprising the step of administering to a mammal an effective amount of the composition comprising ascorbic acid, lysine, magnesium, cysteine, pyridoxine HCL, riboflavin, folic acid, cyanocobalamin vitamin B12, S-Adenosyl-L-Methionine, choline bitartrate, copper glycinate, epigallocatechin gallate, quercetin, asiatic acid, and pycnogenol.

10. The method of claim 6, wherein the nutritional composition comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

11. The method of claim 7, wherein the nutritional composition comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

12. The method of claim 8, wherein the nutritional composition comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

13. The method of claim 9, wherein the nutritional composition comprising about 500 mg to about 10 g ascorbic acid, about 350 mg to about 15 g lysine, about 14 mg to about 750 mg magnesium, about 72 mg to about 2 g cysteine, about 0.7 mg to about 15 mg pyridoxine HCL, about 0.7 mg to about 70 mg riboflavin, about 0.1 mg to about 5 mg folic acid, about 3.5 μg to about 150 μg cyanocobalamin vitamin B12, about 10 mg to about 1 g S-Adenosyl-L-Methionine, about 20 mg to about 2 g choline bitartrate, about 0.7 mg to about 7 mg copper glycinate, about 125 mg to about 525 mg epigallocatechin gallate, about 10 mg to 1 g quercetin, about 70 mg to about 1.5 g asiatic acid, and about 0.7 mg and about 70 mg pycnogenol.

14. The method according to claim 6, wherein the nutritional composition comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

15. The method according to claim 7, wherein the nutritional composition comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

16. The method according to claim 8, wherein the nutritional composition comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

17. The method according to claim 9, wherein the nutritional composition comprising 700 mg ascorbic acid, 800 mg lysine, 21 mg magnesium, 100 mg cysteine, 3 mg pyridoxine HCL, 3 mg riboflavin, 0.4 mg folic acid, 6 μg cyanocobalamin vitamin B12, 100 mg S-Adenosyl-L-Methionine, 180 mg choline bitartrate, 1.5 mg copper glycinate, 175 mg epigallocatechin gallate, 250 mg quercetin, 350 mg asiatic acid, and 3 mg pycnogenol.

18. A method of prevention and treatment resulting in optimization of the composition of connective tissue in mammals.
